(19) United States
(12) Patent Application Publication
(10) Pub. No.: US 2003/0190289 A1
(43) Pub. Date: Oct. 9, 2003

(54) Formulations containing a glucocorticoid drug for the treatment of bronchopulmonary diseases

(76) Inventors: David Lewis, Parma (IT); David Ganderton, Parma (IT); Brian Meakin, Parma (IT); Gaetano Brambilla, Parma (IT); Alessandra Ferraris, Parma (IT)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/275,891

(22) PCT Filed: May 8, 2001
(86) PCT No.: PCT/EP01/05211

(30) Foreign Application Priority Data
May 12, 2000 (IT) MI2000A001051

(51) Int. Cl. A61K 31/573; A61L 9/04

(52) U.S. Cl. 424/45; 514/179

(57) ABSTRACT

The invention discloses formulations for administration through pressurized metered dose inhalers containing as active ingredient a glucocorticoid, in particular the (22R) epimer of budesonide, in solution in a hydrofluorocarbon propellant, a cosolvent and a suitable additive, and their use in the treatment of asthma and other bronchopulmonary disorders.

The present invention relates to formulations to be used in pressurized metered dose aerosol inhalers containing as active ingredient a glucocorticoid in solution in a hydrofluorocarbon propellant, a cosolvent and a suitable additive. In particular the invention relates to formulations containing the (22R) epimer of budesonide in solution, in which the concentration of active ingredient corresponds to single doses of at least 70 μg, preferably of at least 75 μg, even more preferably comprised between 80 and 100 μg. “Single dose” means the amount of active ingredient delivered by a single actuation of the inhaler.

The formulations of the invention are particularly useful for the treatment of asthma and other bronchopulmonary disorders.

The formulations of the invention use a hydrofluoroalkane as a propellant.

It is in fact known that, according to the Montreal Protocol on Substances that Deplete the Ozone Layer, the chlorofluorocarbon propellants such as Freon 11 and Freon 12 are being phased out and also their use in medicinal formulations, although temporarily exempted, will be banished.

In this scenario, hydrofluoroalkanes (HFAs) and in particular 1,1,1 ,2-tetrafluoroethane (HFA 134a) and 1,1,1,2,3,3,3-heptafluoropropane (HFA 227) have been acknowledged to be the best candidates as substitutes for CFCs.

The effectiveness of an aerosol device, particularly a pressurized metered dose aerosol, is a function of the dose deposited in the peripheral tract of the pulmonary tree, that is in turn mainly affected by the particle size distribution (quantified by measuring a characteristic equivalent sphere diameter, known as mass median aerodynamic diameter (MMAD). Particles having a diameter ranging from 0.8 to 5 microns (μm) are usually considered respirable, i.e. capable of being deposited into the lower airways.

In the suspension formulations, the size distribution of the delivered particles almost exclusively depends on the particle size distribution of the suspended particles, and hence on the process used for preparing them (milling or precipitation). Any kind of adjustments of the particle size of the delivered aerosol can be carried out by those skilled in the art, by suitably changing amounts and types of excipients, surface tension of the propellant, size of the metering chamber and diameter of the actuator orifice. However, if the suspended drug has the slightest solubility in propellant, a process known as Ostwald Ripening can lead to particle size growth. Also particles may have tendency to aggregate, or adhere to parts of the MDI, e.g. canister or valve. The effect of Ostwald Ripening and particularly of drug aggregation and hence deposition may be particularly severe for suspension of potent drugs which either need to be formulated in low doses.

Solution compositions provide a number of advantages in that they are easier to be prepared and may avoid the physical stability problems linked to the suspension formulations. However, compared with the latter ones, such formulations can give rise to more severe problems of chemical instability. Furthermore, since the suspended particles no longer contribute to the total volume, the problem of ensuring a direct relationship between increase in dosage and increase in the drug amount deposited at the therapeutical site (respiratory tract) is even more dramatic. The preparation of homogeneous solution formulations requires indeed the addition of cosolvents such as ethanol which, due to their vapor pressure higher than that of the propellant, increase, proportionally to their concentration, the velocity of the aerosol droplets leaving the actuator orifice and hence the fraction of those particles which deposit into the oropharyngeal tract. Therefore, the higher is the dosage of the drug or equivalently the lesser the solubility of the drug, the higher is the amount of cosolvent required to the detriment of the percentage of respirable, and thus therapeutically effective, particles.

In WO 98/56349 the Applicant disclosed solution compositions for use in an aerosol inhaler, comprising an active ingredient, a propellant containing a hydrofluoroalkane (HFA), a cosolvent and further comprising a low volatility component to increase the mass median aerodynamic diameter (MMAD) of the aerosol particles on actuation of the inhaler.

It has now been found that the use of a low volatility component with suitable solvent power for the active ingredient, allows to adjust the amount of cosolvent, specifically ethanol, to be added to the formulation, hence avoiding the negative effects on the therapeutically effective respirable fraction connected with an increase in the cosolvent relative percentage.

Budesonide is a non- halogenated glucocorticosteroid which exhibits a high ratio of topical to systemic activity compared with ohter corticosteroids. The drug is a 1:1 mixture of 2 epimers, designated 22R and 22S (hereinafter referred to as rac-BUD).

As far as the (22R) epimer of budesonide is concerned, (hereinafter referred to as 22R-BUD), studies in the animals evidenced that it is 2 to 3 times more potent than the corresponding (22S) epimer and has a different pharmacokinetic and metabolic profile (Clissold et al. Drugs 1984, 28, 485; Edbäcker et al Drug Metab Disp 1987, 15, 403). A comparison between rac-BUD and its two epimers indicated a rank order of topical activity of 22R >rac-BUD >22S (Clissold et al., ibidem).

Nevertheless, such compound has never been used in therapy neither administered in the form of aerosol for pulmonary delivery, despite to the fact that a higher ratio of local anti-inflammatory to systemic activity might be expected to offer an advantage in terms of systemic tolerability.

In particular, aerosol formulations have never been reported which might be considered as bio-equivalent to the suspension formulations containing rac-BUD currently on the market for the treatment of asthma and related diseases in adults at a single dose of 200 μg.

In order to determine the suitable dose of 22R-BUD for having bio-equivalent formulations, the potency data reported in the literature are not enough, and information about systemic exposure needs to be acquired as well in view of the potential toxicological concerns. Studies carried out by the applicant have indeed confirmed that the systemic exposure of 22R-BUD is different from that of its corresponding epimer being also affected from the characteristics of the formulation as systemic exposure is generally higher for solutions than suspensions.

The preparation of 22R-BUD based aerosol solution formulations able of giving rise to adequate fine particle fraction is also complicated by the fact that 22R-BUD is significantly less soluble than its corresponding epimer either in ethanol and mixtures containing ethanol and HFA134a or ethanol and HFA227.

Without being limited by theory, its lower solubility may be attributed to the higher crystal lattice energy as demonstrated by its melting point, i.e. 275-240°C., which is considerably higher than that of the other epimer (237-240° C.).

Moreover, it has been found that 22R-BUD shows a higher tendency to exhibit chemical degradation than its corresponding epimer, making more problematic the preparation of solution formulations of adequate shelf-life.

In consideration of all problems outlined, it would be highly advantageous to provide an aerosol solution formulation of adequate chemical and physical stability which is able of delivering a therapeutically effective amount of 22R- BUD, by contemporaneously giving rise to plasma levels corresponding to a safe systemic exposure.

In particular, it would be highly advantageous to provide a 22R-BUD-based aerosol solution formulation to be considered as bio-equivalent to the suspension formulations containing rac-BUD currently on the market for the treatment of asthma and related diseases in adults.

In the prior art WO 99/64014 generally claims the use of the (22R) epimer of budesonide in combination with another active ingredient in aerosol pharmaceutical compositions in the form of powders or dose metered aerosols, but no formulation examples are provided.

In WO 00/30608 published on Jun. 2, 2000, it has been proved that 22R-BUD is stable in solution in HFA propellant containing ethanol and optionally a low volatility component, when stored in inhalers having the internal surface consisting of stainless steel, anodized aluminum or lined with epoxy phenol resins.

In the application it is reported a composition containing 48 mg of 22R-BUD in 12 ml HFA 134a, (i.e. 0.4% w/v, which equates to 0.4 g of 22R-BUD per 100 ml of formulation) in the presence of 15% w/w ethanol and 1.3% w/w glycerol. Said formulation contains such high 22R-BUD concentration only for analytical purposes, i.e. for demonstrating that no interconversion from one epimer to the other takes place and is not suitable for threapeutic use.

In the same application, further 22R-BUD solution compositions in HFA 134a or 227 are described whose relevant respirable dose and the respirable fraction have been determined. In these compositions, the concentration of the active ingredient is comprised between 0.06% and 0.14% w/v equivalent to single doses of w 60, 63 and 70 μg, respectively. However, although said formulations give rise to good performances in term of respirable fraction due to the low amount of ethanol, the polarity of the whole solvent system constituted of 7-8% w/w of ethanol and 0.9% w/w of isopropyl myristate or PEG 400 is too low, leading to physical stability problem, i.e. partial precipitation of the active ingredient, after storage under stress conditions.

Moreover, single doses up to 70 μg are considered too low for a suitable therapeutical use.

On the other hand, 22R-BUD has solubility problems in HFA propellants so that the higher the dose, the higher is the amount of cosolvent, preferably ethanol, necessary to dissolve the active ingredient. Ethanol in its turn induces a decrease in the respirable dose, or fine dose, expressed as amount of active particles of size below 4.7 μm, and hence in the respirable fraction, expressed by the ratio between respirable dose and the emitted dose.

For providing physically stable aerosol solution formulations containing suitable concentration of 22R-BUD able of delivering therapeutically effective single doses, it turns out to be necessary to increase the polarity of the whole solvent system by contemporaneously limiting the relative amount of ethanol.

It has now been found, and this is the object of the present invention, that, by suitably selecting the additive, as well as and the relative amounts of the cosolvent and the additive, it is possible to prepare solution compositions containing 22R-BUD in HFA physically and chemically stable after long-term storage, which are able of delivering a therapeutically effective amount of the active ingredient, by contemporaneously giving rise to plasma levels corresponding to a safe systemic exposure.

In order to fulfill the therapeutical requirements, the concentration of 22R-BUD should be equivalent to single doses of 75-100 μg, preferably 80 μg and the amount of ethanol should be adjusted in such a way as to have a respirable fraction of at least 30%, preferably of at least 35%, more preferably of at least 40%.

### DESCRIPTION OF THE INVENTION

The aim of the invention is to provide formulations containing a concentration comprised between 0.12% and 0.20% w/v of the (22R) epimer of budesonide in solution in a HFA propellant, to be used with pressurized metered dose aerosol inhalers for the treatment of bronchopulmonary diseases, said formulations being chemically stable and capable of:

i) delivering a single dose comprised between 75 and 100 μg and preferably of at least 80 μg;

ii) providing a respirable fraction of at least 30%, preferably 35%, more preferably 40%;

iii) giving rise to a clear solution at 4° C. on long-term storage.

iv) giving rise to plasma levels corresponding to a safe systemic exposure.

This object is attained by preparing the formulations of the invention in a carrier consisting of a HFA propellant, a cosolvent, preferably ethanol, and a low volatility component also having solvent properties.

In particular, this object is attained by using a carrier consisting of HFA 134a as propellant, and an amount of ethanol comprised between 10% and 15% w/w in the presence of a suitable additive having low volatility component characteristics as well as solubilizing properties.

Due to such features, the formulations of the invention are therapeutically preferable as they provide the administration of a suitable dose of active ingredient at the action site.

The active is preferably the (22R) epimer of budesonide in such a concentration as to deliver a single dose comprised between 75 and 100 μg, preferably 80 μg. Advantageously, the additive/low volatility component has vapor pressure at 25° C. not above 0.1 kPa, preferably not above 0.05 kPa. Particularly suitable for the use of the invention are additives with a dielectric constant higher than 30, preferably 40 or a dipole moment of at least 1.5, preferably higher than 2 such as glycols and esters, in particular selected from propylene glycol, polyethylene glycol, isopropyl myristate and most preferably glycerol. However, the invention also comprises all substances, alone or in admixture, having similar vapor pressure and polarity characteristics for the active ingredients belonging to this class of drugs. The composition will advantageously contain at least 0.2%, preferably 0.5%, more preferably at least 1%, even more preferably between 1% and 2% w/w of said component.

The cosolvent has advantageously higher polarity than the propellant and is preferably an alcohol, more preferably ethanol. The cosolvent amount in the composition is at least 10% w/w, but it does not exceed 15% w/w and it is preferably 13% w/w. The ratio among the active ingredient, the co-solvent and the additive, expressed as w/v:w/w:w/w, is comprised between 1:50:5 and 1:125:17, preferably between 1:70:6 and 1:110:10, even more preferably 1:80:8.

Preferred hydrofluoroalkane propellants are HFA 134a, HFA 227 or mixtures thereof.

The formulations of the invention are preferably stored in metered dose aerosol inhalers, part or all of their inner metallic surfaces being made of stainless steel, anodized aluminum or lined with an inert organic coating. It has, in fact, been observed that in this type of cans the active ingredient in solution remains chemically stable in time.

The inhalers are advantageously equipped with an actuator with orifice diameter from 0.20 to 0.50 mm, preferably 0.25 mm. The metering chamber has advantageously a volume of at least 50 μl, preferably from 50 to 100 μl.

As a rule, the increase in the volume of the metering chamber negatively affects the fine particle fraction and hence the respirable fraction of the delivered formulation.

It has been found that the formulations of the invention make it possible to use even a valve with a volume of the metering chamber above 50 μl , while keeping the respirable fraction high.

Finally, the invention relates to the use of said formulations in the treatment of bronchopulmonary diseases.

In the following, specific embodiments of the invention are disclosed by way of example.

### EXAMPLE

The aerosol compositions of the invention described below were prepared by the following method. The required components of a composition were added into a can in the following order: drug, low volatility component, absolute ethanol. After crimping the valve on to the can, the propellant was added through the valve.

The weight gain of the can after each component had been added was recorded to allow for the weight percentage of each component in the formulation to be calculated.

The following compositions were prepared.

| a) Composition 1 | | |
|---|---|---|
| (22R)-budesonide | 0.15% | w/v (18 mg/can) |
| ethanol | 13% | w/w |
| glycerol | 1.3% | w/w |

| | |
|---|---|
| HFA 134a | up to 12 ml/can |

Said composition was distributed in inhalers equipped with metering chamber volumes of 50 μl, and actuators with orifice diameter of 0.25 mm.

| b) Composition 2 | | |
|---|---|---|
| (22R)-budesonide | 0.12% | w/v (14.25 mg/can) |
| ethanol | 12% | w/w |
| glycerol | 1.0% | w/w |

| | |
|---|---|
| HFA 134a | up to 12 ml/can |

Said composition was distributed in inhalers equipped with a metering chamber volume of 63 μl and actuators with orifice diameter of 0.25 mm

| c) Composition 3 | | |
|---|---|---|
| (22R)-budesonide | 0.16% | w/v (19.2 mg/can) |
| ethanol | 13% | w/w |
| glycerol | 1.3% | w/w |

| | |
|---|---|
| HFA 134a | up to 12 ml/can |

Said composition was distributed in inhalers equipped with metering chamber volume of 50 μl and actuators with orifice diameter of 0.25 mm.

The aerodynamic particle size distribution of the tested formulations was determined using a Multistage Cascade Impactor according to the procedure described in the European Pharmacopoeia 2nd edition, 1995, part V.5.9.1. pages 15-17.

In this specific case an Andersen Cascade Impactor (ACI) was used.

Results were obtained as a mean of 2 cans. For each device, 5-25 cumulative actuations were carried out after discarding the first 5.

Deposition of the drug on each ACI plate was determined by high pressure liquid chromatography (HPLC).

MMAD values were calculated from plots of the cumulative percentage undersize of drug collected on each ACI plate (probit scale), against the upper cut off diameter for each respective ACI plate (log10 scale).

The fine particle dose (respirable dose) of each formulation was determined from the mass of drug collected on Stages 3 through to Filter, namely particles of diameter <4.7 μm, divided by the number of actuations per experiment.

The delivery characteristics of the formulations are reported in Tables 1, 2 and 3. The following parameters were determined: the metered dose, which is the sum of the dose delivered -through the device plus the active ingredient residue deposited on the device actuator; the delivered dose, which is the amount of active particles deposited on the various ACI stages; the fine particle dose or respirable dose which is the amount of active particles of size less than 4.7 μm; the fine particle fraction or respirable fraction which is the ratio between the respirable dose and the delivered dose.

It is evident that all the formulations give rise to very good performances in term of respirable fraction.

TABLE 1
Performances of composition 1 (MMAD = 2.6-2.7 μm)

| Nominal | Respirable | Respirable | Metered | Delivered |
|---|---|---|---|---|
| dose | dose | fraction | dose | dose |
| (μg) | (μg) | (%) | (μg) | (μg) |
| 75 | 39.2 | 56.2 | 74.7 | 69.7 |

Nominal dose: theoretical dose per single actuation

TABLE 2
Performances of composition 2 (MMAD = 2.5 μm)

| Nominal | Respirable | Respirable | Metered | Delivered |
|---|---|---|---|---|
| dose | dose | fraction | dose | dose |
| (μg) | (μg) | (%) | (μg) | (μg) |
| 75 | 40.5 | 57.1 | 75.7 | 70.9 |

TABLE 3

| Nominal | Respirable | Respirable | Metered | Delivered |
|---|---|---|---|---|
| dose | dose | fraction | dose | dose |
| (μg) | (μg) | (%) | (μg) | (μg) |
| 80 | 37.9 | 50.3 | 80.9 | 75.5 |

1. A pharmaceutical formulation to be used in a metered dose aerosol inhaler, comprising the (22R) epimer of budesonide as active ingredient in a concentration comprised between 0.12% and 0.20% w/v in solution in a mixture consisting of a hydrofluoroalkane propellant, a cosolvent and a low volatility component.

2. A formulation as claimed in claim 1, wherein the low volatility component has a dielectric constant of at least 30, preferably at least 40 or a dipole moment of at least 1.5, preferably at least 2.

3. A formulation as claimed in claims 1 and 2, wherein the low volatility component is a glycol, selected from propylene glycol, polyethylene glycol and glycerol.

4. A formulation as claimed in claims 1-3, wherein the propellant is HFA 134a, the low volatility component is glycerol and the cosolvent is ethanol.

5. A formulation as claimed in claims 1-4, wherein the amount of ethanol is comprised between 10% and 15% w/w and that of glycerol is at least 1% w/w.

6. A pharmaceutical formulation as claimed in claims 1-5 wherein the respirable fraction is at least 30%, preferably 35%, more preferably 40%.

7. A formulation as claimed in claims 1-6 wherein the single dose of the active ingredient is at least 70 μg.

8. A pharmaceutical formulation as claimed in claims 1-7 to be used in pressurized metered dose aerosol inhalers, in the treatment of bronchopulmonary diseases.

9. A formulation as claimed in any one of claims 1 to 8, contained in metered dose aerosol inhalers having part or all of the inner metal surfaces made of anodized aluminum, stainless steel or lined with an inert organic coating.
